(19) United States
(12) Patent Application Publication
(10) Pub. No.: US 2025/0256005 A1
(43) Pub. Date: Aug. 14, 2025

(54) FLOWABLE PASTE WITH ELLAGIC ACID-NICKEL COMPLEX AND USES THEREOF FOR ACTIVATING COAGULATION

(71) Applicants: Ethicon, Inc., Somerville, NJ (US); Omrix Biopharmaceuticals Ltd., Kiryat-Ono (IL)

(72) Inventors: Ashley DeAnglis, Skillman, NJ (US); Guanghui Zhang, Belle Mead, NJ (US); Benjamin David Fitz, Port Murray, NJ (US); YI-Lan Wang, Hillsborough, NJ (US); Samar Azizighannad, Nutley, NJ (US); Brian Scott Aitken, Jacksonville, FL (US); Gal Dela, Ness Ziona (IL)

(21) Appl. No.: 19/007,726

(22) Filed: Jan. 2, 2025

Related U.S. Application Data
(60) Provisional application No. 63/553,175, filed on Feb. 14, 2024.

(51) Int. Cl. A61L 26/00

(52) U.S. Cl. CPC A61L 26/0033; A61L 26/0004; A61L 26/0038; A61L 26/009; A61L 2300/418

(57) ABSTRACT

The invention relates to hemostatic compositions comprising ellagic acid-metal complex and uses thereof for activating the intrinsic pathway of blood coagulation. In particular, disclosed are hemostatic compositions comprised of ellagic acid complexed with nickel ions, the complex being dispersed or embedded within an absorbable polymeric matrix such as gelatin. Further disclosed are method for making the hemostatic compositions.

### CROSS-REFERENCE TO RELATED APPLICATION

This application claims the benefit of U.S. Application No. 63/553,175 filed Feb. 14, 2024.

### FIELD OF THE INVENTION

The present invention relates, inter alia, to hemostatic compositions comprising ellagic acid-nickel complex and uses thereof for activating coagulation.

### BACKGROUND OF THE INVENTION

Topical hemostatic agents have become essential tools to minimize bleeding in surgical or emergency settings and to reduce the associated risks of serious complications. Adjunctive hemostats used by surgical teams may contain a wide variety of agents such as gelatins, fibrin sealants, oxidized regenerated cellulose (ORC), or synthetic hydrogels and adhesives. Products based on animal (e.g., porcine and bovine) gelatins and plant-derived ORC are at the forefront of bleeding management because they can be used in a variety of surgical procedures and because of their low cost.

Flowable gelatin-based products are topical hemostats consisting of matrices that form viscous gels capable of entering and coating difficult to assess spaces. To improve the efficacy of these gelatin-based matrices and to accelerate coagulation in situ, thrombin, a terminal serine protease of the coagulation cascade, is often added to the paste.

While human plasma derived thrombin is generally considered safe, and approved by the U.S. Food and Drug Administration (FDA) and other regulatory authorities, it is purified from human plasma and thus there is a theoretical risk of disease transmission. Additionally, the cost of the thrombin (even as little as 2000 IU) significantly increases the cost of the gelatin hemostat. Finally, the thrombin (which is an active enzyme) is generally supplied as a lyophilized powder which must be reconstituted prior to use. If thrombin can be omitted from the formulation, both the stability and ease of preparation of the gelatin paste could be improved.

Ellagic acid (EA) is a natural phenol antioxidant found in numerous fruits and vegetables and is available as a dietary supplement. EA can be complexed with various metal ions and has been used for in vitro coagulation assays for many decades (Bock et al., Biochemistry 1981, 20, 25, 7258-7266) to provide a rapid activator of the intrinsic pathway, sometimes referred to as “the contact activation pathway”. The main use of Ellagic acid Nickel complex was for the activated partial thromboplastin time (aPTT). For example, the use of EA complexed with metal ions to improve upon existing assays, e.g., the aPTT, which utilize less sensitive and consistent activators of the intrinsic pathway was previously disclosed e.g., in EP0525035B1.

U.S. Pat. No. 5,709,889A discloses novel assays and reagents for determining coagulative properties of blood or plasma, as are agents and methods for stemming bleeding. The novel ellagic acid-based activators consist essentially of aqueous solutions of ellagic acid, phenol and suitable metal ions and give defined values for a novel platelet assay. Inventive coagulation reagents including propyl gallate or tannin are also disclosed, as are aPTT reagents having sensitivity to heparin and Factor deficiencies far superior to prior aPTT reagents.

U.S. Pat. No. 5,041,558A relates to an accelerator of the activity of hydrolase, especially one that accelerates the activation of the precursors of serine protease, including blood coagulation Factor XII, and that accelerates the enzyme activity of serine protease produced by the activation of the said precursors. Moreover, this invention relates to an accelerator of blood coagulation with the use of the said accelerator.

EP3258974A1 discloses hemostatic composition, comprising water-retaining, binder dust suppression, inorganic and organic hemostatic agents, and hemostatic device comprising the composition of hemostatic agents and a container that keeps hemostatic composition.

EP1011727A1 discloses hemostatic product of the invention is active in all patients including those treated with heparin. It consists of a viscous, biologically compatible, biodegradable composition and/or capable of being biologically eliminated but which is not a collagen composition, in which is contained a hemostatic extract of snake venom, for instance batroxobin or ancrod. The viscous composition is formed in particular from hyaluronic acid, optionally esterified.

U.S. Pat. No. 7,371,722B2 relates to a pharmaceutically active substance for producing a drug that is capable of generating thrombin or that contains thrombin and compositions comprising thereof. The pharmaceutically active substance contains (A) prothrombin (coagulation factor II) obtained from plasma or by genetic engineering, (B) coagulation factors V, VIII, IX, X obtained from plasma or by genetic engineering that at least partially may be present in their activated state, and coagulation factor XIa obtained from plasma or by genetic engineering, and (C) prion-safe, coagulation-promoting phospholipids, where the phospholipids are optionally contained in liposomes.

WO2009098029A2 relates to wound pads or wound dressing articles that can promote or accelerate blood coagulation.

CN107281472A relates to medical material field, more particularly, to a kind of medical nano-silver antibacterial temperature-sensitive hydrogel and preparation side method.

Marcińczyk et al. (Front Pharmacol. 2022 Jan. 13;12:806891) relates to tannins as hemostasis modulators.

### SUMMARY OF THE INVENTION

The present invention relates, inter alia, to comprising ellagic acid (EA)-nickel complex and uses thereof for activating coagulation. An object of the present invention is to provide compositions that are capable of rapidly activating the intrinsic pathway of coagulation.

The present invention utilizes a combination of gelatin paste with a bioactive polyphenolic compound e.g., EA, complexed with nickel ions (EA-Ni) to create a flowable paste that is capable of rapidly activating the intrinsic pathway of coagulation. In vitro assays utilizing whole blood were used to optimize the clotting properties of the EA-Ni complex alone and in combination with gelatin, and to demonstrate the mechanism of action. In vivo studies were conducted to compare the performance of this novel EA-Ni gelatin paste and standard gelatin paste with and without thrombin. The in vivo studies demonstrated that comparable hemostatic performance could be achieved with EA-Ni gelatin paste versus gelatin paste with thrombin. The flowability and consistency of the EA-Ni gelatin paste were similar to the gelatin formulations without the EA. A broad range of EA-Ni activity was demonstrated. In addition, the amount of nickel required for rapid clotting was so low that it was below the level of concern from a toxicity standpoint. SURGIFLO®/SURGIFOAM™ mixture with Ellagic Acid-Nickel Complex may yield rapid in-vitro clotting times, as well as fast/effective hemostasis in an in-vivo spleen biopsy model. Also is shown that there may be some optimized composition with specific buffers.

According to an aspect of the present invention, there is provided a hemostatic composition comprising a bioactive polyphenolic compound, e.g., ellagic acid (EA), complexed with metal ions, the complex being dispersed, or embedded within an absorbable polymeric matrix.

In some embodiments, the polymeric matrix comprises collagen or a derivative thereof. In some embodiments, the derivative of the collagen comprises gelatin. In some embodiments, the gelatin is in a lyophilized or dried form.

In some embodiments, the metal is selected from the group consisting of nickel, cobalt and copper. In some embodiments, the metal is nickel.

In some embodiments, the metal ions and the EA are present at a molar ratio ranging from 100:1 to 1:100 metal to EA, optionally 10:1 to 1:10 molar ratio of metal ions to EA.

In some embodiments, the composition or the absorbable polymeric matrix e.g., gelatin is in the form selected from a flowable paste at room temperature, a sponge-like matrix, a powder, and a combination thereof.

In some embodiments, the composition the composition or the absorbable polymeric matrix is in the form of a paste.

In some embodiments, the paste is characterized by a viscosity ranging from 1000 to 5000 Pa.s.

In some embodiments, the matrix comprises a combination of gelatin in the paste form and in the powder form.

In some embodiments, the composition is substantially devoid of one or more coagulation factors selected from the inactive or active forms of Factors II, V, VII, VIII, IX, X, XI, XII and fibrinogen. In some embodiments, the composition is substantially devoid of thrombin.

In some embodiments, the metal ions are present at a preferred concentration ranging from 11,200:1 to 7:1, w/w, optionally, 1120:1 to 70:1 matrix, e.g., gelatin, to metal ion.

In some embodiments, the EA complex is present at a concentration ranging from 56,000:1 to 35:1, w/w or 5600:1 to 350:1, w/w, optionally, by 5600:1 to 350:1, w/w, matrix, e.g., gelatin to EA-Ni.

According to another aspect of the present invention, there is provided a method of treating a subject having a disease or an injured tissue that can benefit from rapidly activating the intrinsic pathway of blood coagulation, the method comprising administrating to the subject a therapeutically effective amount of ellagic acid (EA) complexed with metal ions, said complex being incorporated within a matrix comprising collagen or a derivative thereof.

In some embodiments, the administrating comprises contacting the injured tissue with said matrix.

In some embodiments of this aspect, the metal is nickel.

In some embodiments, a blood clotting is affected within less than about 400 sec, upon the contacting.

In some embodiments of this aspect, the collagen derivative comprises gelatin in the form selected from a flowable paste at room temperature, a sponge-like matrix, a compressed tablet, a powder and any combination thereof.

In some embodiments of this aspect, the matrix comprises a combination of gelatin in the paste form and in the powder form.

According to another aspect of the present invention, there is provided method of making a hemostatic composition comprising ellagic acid (EA) complexed with metal ions to an absorbable polymeric matrix, comprising the step of: adding to the matrix said complex at a ratio ranging from 56,000:1 to 35:1, or 5600:1 to 350:1 w/w, polymeric matrix to EA-Ni complex, by weight, respectively.

In some embodiments of this aspect, the polymeric matrix comprises collagen or a derivative thereof.

In some embodiments of this aspect, the derivative of the collagen comprises gelatin.

In some embodiments of this aspect, the metal is nickel.

In some embodiments of this aspect, the matrix, e.g., gelatin, is in the form selected from a flowable paste a at room temperature, a sponge-like matrix, a compressed tablet, a powder, and a combination thereof.

In some embodiments of this aspect, there is provided a hemostatic composition obtainable by this method.

According to another aspect of the present invention, there is provided a kit comprising: a container containing ellagic acid (EA) complexed with metal ions; a polymeric matrix; an applicator for applying the composition to a tissue; and optionally instructions for use.

In some embodiments of this aspect, the polymeric matrix comprises collagen or a derivative thereof.

In some embodiments of this aspect, the derivative of the collagen comprises gelatin.

In some embodiments of this aspect, the metal is nickel.

In some embodiments of this aspect, the matrix, e.g., gelatin, is in the form selected from a flowable paste a at room temperature, a sponge-like matrix, a powder, and a combination thereof.

In some embodiments of this aspect, the EA complexed with metal ions is provided within a buffer.

In some embodiments of this aspect, the buffer comprises a solution having tetramethylammonium hydroxide (TMAH).

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

In order to better understand the subject matter that is disclosed herein and to exemplify how it may be carried out in practice, embodiments will now be described, by way of non-limiting example only, with reference to the accompanying drawings, in which:

FIG. 1 presents a photographic image showing recalcified blood after several minutes with Celite particles, 12 mg, (clotted in tube; top) and bioactive glass (45 mg).

FIG. 2 present graphs showing comparative Activated Clotting Time (ACT) data for Celite (12 mg) and bioactive glass (45 mg).

FIG. 3 presents a graph showing ACT Time Course with celite, showing the time course over a day's time testing celite (12 mg) over the course of 6 h in the ACT analyzer.

FIG. 4 presents a graph showing ACT data with varying Celite concentrations.

FIG. 5 presents a bar graph showing ACT data with various types of polyphosphate and Celite.

FIG. 6 presents a bar graph showing comparative ACT data shown in FIG. 2, versus results for the Ellagic Acid (EA)-Ni complex, and for no activator.

FIGS. 7A-7B present graphs showing a dose response study with respect to blood clotting time performed EA-Ni complex: mean values of triplicate results at each concentration tested (FIG. 7A), and a concentration range of the complex in which there is no significant trend in clotting times (FIG. 7B).

FIG. 8 presents a bar graph showing the clotting rate impact of phospholipids (Cephalin) added to EA-Ni complex.

FIG. 9 presents a bar graph showing the clotting rate impact of EA-Ni complex with added corn trypsin inhibitor (CTI), an inhibitor of factor XIIa.

FIG. 10 presents a bar graph showing comparative results of hemostatic efficacy on an in vivo porcine spleen bleeding model of various preparations.

FIG. 11 presents a bar graph showing the clotting rate impact of various buffers added to a formulation of SURGIFLO® gelatin with EA-Ni complex.

FIG. 12A-12B present photographic images captured for tablets made of gelatin sponges before (left panels) and after (right panels) soaking in EA-Ni complex, for uncompressed tablets (FIG. 12A), and for compressed tablets (FIG. 12B).

FIG. 13 presents a bar graph showing comparative results of mean clotting time of various gelatin tablets as described in Example 3.

### DESCRIPTION OF EMBODIMENTS OF THE INVENTION

The present invention utilizes a combination of gelatin paste with EA complexed with nickel ions (EA-Ni) to create a flowable paste that is capable of rapidly activating the intrinsic pathway of coagulation. In vitro assays utilizing whole blood were used to optimize the clotting properties of the EA-Ni complex alone and in combination with gelatin, and to demonstrate the mechanism of action. In vivo studies were conducted to compare the performance of this novel EA-Ni gelatin paste and standard gelatin paste with and without thrombin. The in vivo studies demonstrated that comparable hemostatic performance could be achieved with EA-Ni gelatin paste versus gelatin paste with thrombin. The flowability and consistency of the EA-Ni gelatin paste were similar to the gelatin formulations without the EA.

A broad range of EA-Ni activity was demonstrated. In addition, the amount of nickel required for rapid clotting was so low that it was below the level of concern from a toxicity standpoint. SURGIFLO®/SURGIFOAM™ mixture with Ellagic Acid-Nickel Complex may yield rapid in-vitro clotting times, as well as fast/effective hemostasis in an in-vivo spleen biopsy model. Also is shown that there may be some optimized composition with specific buffers.

An object of the present invention is to provide a composition which may be easily applied to a bleeding site of need, especially in difficult-to-reach areas of the body. A further advantage of the compositions of the invention is that they are bioabsorbable, and therefore may be left behind following surgery without causing any side effects.

Before explaining at least one embodiment of the invention in detail, it is to be understood that the invention is not necessarily limited in its application to the details set forth in the following description or exemplified by the Examples. The invention is capable of other embodiments or of being practiced or carried out in various ways.

### The Composition

According to an aspect of some embodiments of the present invention, there is provided a hemostatic composition comprising ellagic acid (EA) complexed with metal ions. In some embodiments, the complex is dispersed, incorporated or embedded within an absorbable polymeric matrix for, e.g., homogenous distribution of the complex therein.

The term “composition” as used herein includes a substance or preparation formed by combination or mixture of various ingredients. In some embodiments, the composition comprises a homogenous suspension, formulation or gel. As used herein, by “homogeneous” it is meant to refer to a uniform composition, texture, or suspension throughout, e.g., having a density and/or viscosity that vary within a range of up to ±25%. In some embodiments, the composition is homogeneous.

The term “complex” refers to a compound comprising one or more metal cations and one or more anionic or neutral ligands (e.g., EA), with at least one of the ligands being bonded to the metal e.g., by means of donor electrons.

The term “ellagic acid” (EA) or “ellagic acid compound” used herein is a general term which encompasses ellagic acid, a salt of ellagic acid, and a metal chelate compound of ellagic acid. Ellagic acid may be used as an activator of a contact factor involved in the intrinsic coagulation pathway.

Ellagic acid is known to be an activator of a contact factor involved in the intrinsic coagulation pathway. Examples of the contact factors include, but are not particularly limited to, prekallikrein, high-molecular-weight kininogen, factors XII and XI, and the like. The ellagic acid may be natural or synthetic ellagic acid. Examples of the salt of ellagic acid include, but are not particularly limited to, a metal salt of ellagic acid, and the like. The ellagic acid forms a metal chelate compound with a metal ion through a coordinate bond to have a strong activating effect on the contact factor involved in the intrinsic coagulation pathway. Therefore, in the reagent as provided herein, the ellagic acid compound is preferably a metal chelate compound of ellagic acid. In exemplary embodiments, the metal ion is nickel.

In some embodiments, intrinsic coagulation pathway refers to Factor XII (“FXII”) activation (see FIG. 9).

The term “polymeric matrix” as used herein refers to a molecule comprised of several repeating and linked chemical units and may serve as a site where one or more agents are linked, entrapped or embedded thereto. Typically, the polymeric matrix refers to a biocompatible and/or biodegradable polymer.

As used hereinthroughout, the term “polymer” describes an organic substance composed of a plurality of repeating structural units (backbone units) covalently connected to one another. This term also includes co-polymer. The term “co-polymer” as used herein, refers to a polymer of at least two chemically distinct monomeric units.

The term “absorbable polymeric matrix” as used herein is defined as a natural or synthetic, biocompatible polymer (including copolymers) that is absorbable and can degrade and be absorbed e.g., by the body when it is implanted in humans and animals.

More specifically, the term “bioabsorbable,” as used herein, refers to the ability of a tissue-compatible material to degrade in the body after implantation therewithin, and that the referenced material is biocompatible with at least a portion of the material being either excreted, metabolized or assimilated by the body following implantation therewithin (see e.g., Barrows, “Synthetic Bioabsorbable Polymers,” p. 243, In: High Performance Biomaterials—A comprehensive Guide to Medical and Pharmaceutical Applications, Michael Szycher, ed., Technomic Publishing, Lancaster, Pa., 1991).

Absorbable polymers readily break down into small segments when exposed to moist body tissue. The segments then either are absorbed by the body, or passed by the body. More particularly, the biodegraded segments do not elicit permanent chronic foreign body reaction, because they are absorbed by the body or passed from the body, such that no permanent trace or residual of the segment is retained by the body.

The term “embedded” refers to terms as follows: “dispersed”, “incorporated with” or “entrapped”, “encapsulated in”, “coating” “comprises” and otherwise “provided in remote or physical connection with”, or may be considered part of the polymeric structure or the polymeric particles made therefrom.

In some embodiments, the polymeric matrix comprises collagen or a derivative thereof.

In some embodiments, the polymeric matrix comprises gelatin in the form of microparticles. As used herein the term “gelatin” also includes gelatin derivatives such as gelatin derivatized with aromatic sulfonyl chlorides, carboxylic acid chlorides, carboxylic acid anhydrides, aryl isocyanates, etc. The term “gelatin” also includes collagen and derivatives thereof.

The term “derivative” as used herein refers to a substance which comprises the same basic carbon skeleton and functionality as the parent compound, but can also bear one or more substituents or substitutions of the parent compound.

In some embodiments, the matrix, e.g., the gelatin is in the form selected from a flowable paste at room temperature, a sponge-like structure (e.g., matrix or scaffold), a powder, a compressed tablet, and a combination thereof.

In some embodiments, the matrix comprises both gelatin powder and gelatin gel. In exemplary embodiments of the present invention, there is provided a hemostatic composition comprising ellagic acid (EA) complexed with nickel ions, with the complex being dispersed, incorporated or embedded within gelatin powder which is distributed in a gelatin gel.

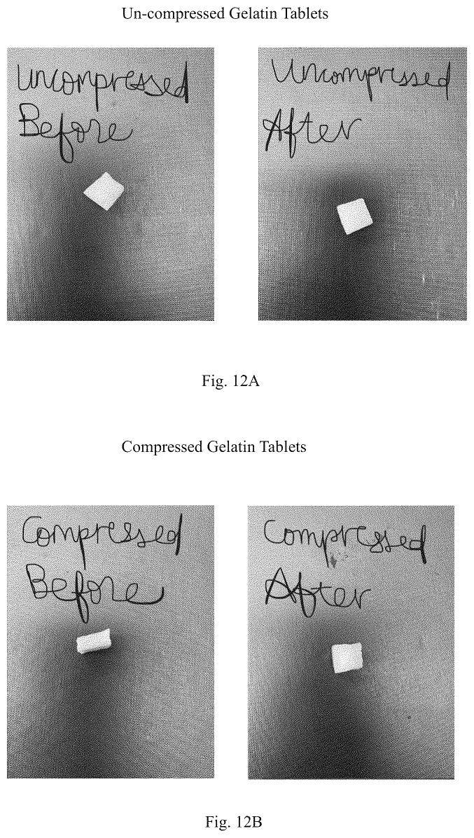

In additional exemplary embodiments of the present invention, there is provided a hemostatic composition comprising ellagic acid (EA) complexed with nickel ions, with the complex being dispersed, incorporated or embedded within a gelatin gel, and an appropriate buffering agent, optionally without the presence of gelatin powder. Non-limiting exemplary buffering agents are selected from CHES and bicarbonate buffers (see FIG. 11).

The term “sponge-like structure”, as used herein, refers to a three-dimensional structure which is flexible, e.g., reversibly compressible foams that are porous, elastic, fibrous, and/or resilient.

The term “powder” refers to a substance containing ground, pulverized, or otherwise finely dispersed solid particles. The term “solid” characterizes the state of the compound or composition at room temperature (e.g., about 25° C.) and at atmospheric pressure (760 mmHg), i.e. a compound or a composition of high consistency which retains its form during storage. This term in the present application also relates to non-fluid particles, or dissolved substance. As opposed to “liquid” compounds and compositions, the solid does not flow under its own weight.

The term “compressed tablet” as referred to herein means a tablet which has been produced by a compression step which results in the production of a dimensionally stable tablet.

In additional exemplary embodiments of the present invention, there is provided a hemostatic composition comprising ellagic acid (EA) complexed with nickel ions, with the complex being incorporated or embedded within a compressed gelatin tablet. Reference is made to FIG. 13 showing that tablets soaked in EA-Ni followed by lyophilization and compression had shorter clotting times than similar uncompressed tablets.

In some embodiments, the composition is in the form of a paste. The term “paste” as used herein, relates to the consistency of the composition at at-least one temperature around the room temperature, and may refer to a fluid mixture of solid particles. The term “paste” according to the present disclosure may also include a slurry. A slurry may functionally be regarded as a thin, watery paste. A paste according to the present disclosure may also include pores comprising of an expandable gas, such as air. Accordingly, the composition is a paste, or is in a paste (or pasty) consistency at around room temperature.

The consistency of the composition may further be divided into a more fluid paste, referred to herein as “flowable”, and a doughier paste, referred to herein as “non-flowable” (or “not flowable”).

Accordingly, in some embodiments, the composition is flowable at at-least one temperature around room temperature, such as at one or more temperature values selected from 10° C., 15° C., 20° C., 25° C., 30° C., 35° C., 37° C., and 40° C. This composition is referred to hereinbelow as the “flowable” composition.

Accordingly, in some embodiments, the composition is non-flowable at at-least one temperature around room temperature, such as at one or more temperature values selected from 10° C., 15° C., 20° C., 25° C., 30° C., 35° C., 37° C., and 40° C. This composition is referred to hereinbelow as the “non-flowable” or “not flowable” composition.

The term “non-flowable” (or “not flowable”) paste is also referred to herein as “putty”. As used herein, the term “flowable” in the context of paste relates to a more fluid consistency at around the room temperature, which may still flow after application of the composition in/on the bleeding site. The terms “non-flowable” or “putty” refer to a doughier consistency at e.g., 37° C., which takes longer to settle, and has a better shape retention than a “flowable” paste.

Although the consistency of the composition e.g., as “paste” is defined above, alternatively, or additionally, the composition may also be defined in terms of resistance under certain conditions, as detailed hereinbelow. Typically, viscosity, the measurement of the resistance of a liquid to flowing.

In some embodiments, the composition has a viscosity of about 1000 to 5000 cP at one or more temperature values selected from 10° C., 15° C., 20° C., 25° C., 30° C., 35° C., 37° C., and 40° C. In some embodiments, the composition has a viscosity of about 2500 to 3500 cP at one or more temperature values selected from 10° C., 15° C., 20° C., 25° C., 30° C., 35° C., 37° C., and 40° C. In some embodiments, the composition has a viscosity of about 1000, about 1500, about 2000, about 2500, about 3000, about 3500, about 4000, about 4500, or about 5000 cP, including any value and range therebetween, at one or more temperature values selected 10° C., 15° C., 20° C., 25° C., 30° C., 35° C., 37° C., and 40° C.

In some embodiments, the composition is sterile. The term “sterile” as used herein means having a low bioburden, effectively being germ-free, e.g., being free from microorganisms, e.g., bacteria and viruses. Sterilization is the process of reducing the bioburden to an effectively germ-free level. A sterile liquid is generally defined as a liquid that underwent sterile filtration. Accordingly, in some embodiment, a sterile composition has been sterilized by sterile filtration, e.g., by passing through a filter of 0.22 μm or less.

In some embodiments, the metal ion is a divalent ion. In some embodiments, the metal ion is selected from zinc, iron and sodium.

In some embodiments, the metal ion is a divalent ion. In some embodiments, the metal ion is selected from nickel, cobalt and copper. In some embodiments, the metal ion is nickel.

In some embodiments, the metal (e.g., nickel) ions and the EA are present at a respective molar ratio ranging from 1:100 to 100:1 or, in some embodiments from 1:10 to 10:1 metal (e.g., nickel) ions to EA. In some embodiments, the metal (e.g., nickel) ions and the EA are present at a molar ratio ranging from about 1:5 to 5:1 metal to EA. In some embodiments, the metal (e.g., nickel) ions and the EA are present at molar of about1: 4 to 4:1 metal to EA. In some embodiments, the metal (e.g., nickel) ions and the EA are present at molar of 4:1, 5:1, 6:1, 7:1, or 8:1 metal to EA, including any value and range therebetween. Typically, but not exclusively, the metal (e.g., nickel) ion is in excess and the complex is kept in a suspension rather than forming a single large network of EA and metal (e.g., nickel) ions. In exemplary embodiments, the metal (e.g., nickel) ions and the EA are present at a respective molar ratio of approximately 1:1, e.g., 0.89:1.

In some embodiments, the metal ions are present at a concentration ranging from 11,200:1 to 7:1, w/w, gelatin to metal ion (e.g., nickel). In some embodiments, the metal ions are present at a concentration ranging from 1120:1 to 70:1, w/w, gelatin to metal ion (e.g., nickel). In some embodiments, the metal ions are present at a concentration ranging from 1120:1 to 7:1, w/w, gelatin to metal ion (e.g., nickel). In some embodiments, the metal ions are present at a concentration ranging from 11,200:1 to 70:1, w/w, gelatin to metal ion (e.g., nickel).

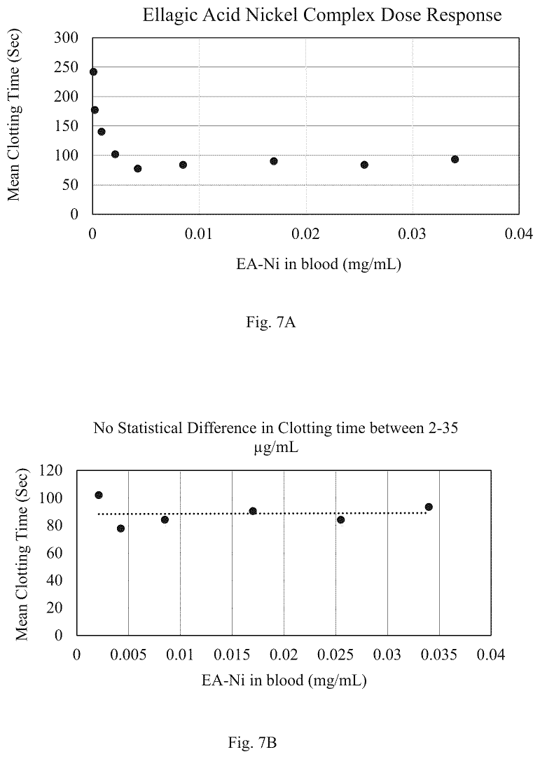

In exemplary embodiments, the EA-Ni may be diluted 1:40 and still accelerate clotting (i.e. less than 200 sec) based on dose response studies (see FIGS. 7A-7B). Calculating the preferred range of the ratio of gelatin to EA-Ni would be 56,000:1 to 35:1. In addition, EA-Ni may be diluted 1:4 to rapidly accelerate clotting (i.e. approximately 100 sec) based on dose response studies. Calculating the preferred range of the ratio of gelatin to EA-Ni would therefore be 5600:1 to 350:1, respectively.

Accordingly, in some embodiments, the EA complex is present at a concentration ranging from 56,000:1 to 35:1, w/w, gelatin to EA-metal ion (e.g., nickel). In some embodiments, the EA complex is present at a concentration ranging from 5600:1 to 35:1, w/w, gelatin to EA-metal ion (e.g., nickel). In some embodiments, the EA complex is present at a concentration ranging from 5600:1 to 350:1, w/w, gelatin to EA-metal ion (e.g., nickel). In some embodiments, the EA complex is present at a concentration ranging from 56,000:1 to 350:1, w/w, gelatin to EA-metal ion (e.g., nickel). In exemplary embodiments, the weight ratio of gelatin to EA-Ni is about 1350:1 to about 1450:1, e.g., 1409:1, w/w.

In some embodiments, the gelatin is in a in a lyophilized form. The term “lyophilized” used herein refers to the preparation of a dry composition formed by rapid freezing and thawing in the frozen state (sometimes referred to as “sublimation”). This process can be done under vacuum at a reduced air pressure resulting from drying at a temperature lower than that required at e.g., atmospheric pressure.

In some embodiments, the matrix comprises a combination of gelatin in the paste form and in the powder form.

In some embodiments, the composition is substantially devoid of enzymes that can activate mammalian coagulation factors (procoagulant enzymes).

The term “coagulation factors” or “procoagulant enzymes” refers to the plasma proteins e.g., enzymes, which interact with platelets in a complex cascade of enzyme-catalyzed reactions, leading to the formation of fibrin for the initiation of a blood clot in the blood coagulation process. This term refers to the components, in the intrinsic, extrinsic and common coagulation pathways. Coagulation factors are generally serine proteases when activated, but also comprise glycoproteins (Factors VIII and V) or other types of enzymes, such as transglutaminase (Factor XIII).

The term “plasma protein” as used herein means any protein, especially any industrially or therapeutically important protein contained in plasma.

Procoagulant enzymes, proteins and peptides may be naturally occurring, recombinant, or synthetic, and may be selected from prothrombin, fibrin, fibronectin, heparinase, Factor X/Xa, Factor VII/VIIa, Factor IX/IXa, Factor XI/XIa, Factor XII/XIIa, tissue factor, batroxobin, ancrod, ecarin, von Willebrand Factor, platelet surface glycoproteins, vasopressin and vasopressin analogs, epinephrine, selectin, procoagulant venom, plasminogen activator inhibitor, platelet activating agents, synthetic peptides having hemostatic activity, derivatives of the above and any combination thereof.

In some embodiments, the composition is substantially devoid of one or more coagulation factors selected from the inactive or active forms of Factors II, V, VII, VIII, IX, X, XI, XII and fibrinogen.

In some embodiments, the composition is substantially devoid of thrombin.

As used herein, “thrombin” denotes an activated enzyme which results from the proteolytic cleavage of prothrombin (factor II). Thrombin may be produced by a variety of methods of production known in the art, and includes, but is not limited to, recombinant thrombin and plasma derived thrombin.

Human thrombin is a 295 amino acid protein composed of two polypeptide chains joined by a disulfide bond. Both human and non-human (e.g., bovine, porcine or salmon) thrombin may be used within the scope of the present disclosure.

As used herein, the term “substantially devoid of” with regard to a component of a composition refers to a component which is present in the composition at a concentration of less than 0.1% w/w of the total composition, or is absent.

In some embodiments, the composition is devoid of thrombin. In some embodiments, the composition is devoid of one or more coagulation factors selected from the inactive or active forms of Factors II, V, VII, VIII, IX, X, XI, XII and fibrinogen.

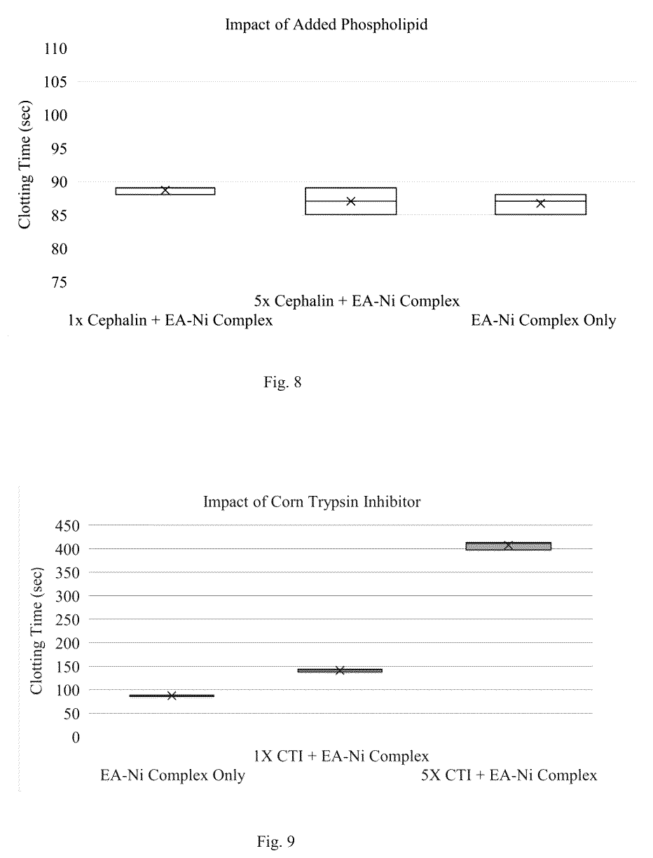

In some embodiments, the composition is devoid of platelets. In some embodiments, the composition is devoid of added phospholipids (see FIG. 8 showing no improvement in clotting time observed with added phospholipid to fresh whole blood).

In some embodiments, the composition, in any embodiment thereof, further comprises an additional pharmaceutically active agent being contained within the composition or on a surface of the composition.

In some embodiments, the pharmaceutically active agent is selected from a therapeutically active agent and a labeling agent.

In some embodiments, the composition, in any embodiment thereof, further comprises an additional pharmaceutically active agent being contained within the composition or on a surface of the composition. In some embodiments, the pharmaceutically active agent is selected from a therapeutically active agent and a labeling agent.

In some embodiments, the therapeutically active agent is selected a stem cell, a growth factor, a bone morphogenetic protein, a cell, a cytokine, a hormone, a medicament, a mineral, a plasmid with therapeutic potential, and a combination thereof. In some embodiments, the therapeutically active agent may be an antifibrinolytic agent, e.g., aprotinin, tranexamic acid (Cyklokapron), epsilon amino caproic acid (Amicar), Nafamostat mesylate, lysine, lysine derivatives, and a combination thereof.

In some embodiments, the composition is stable. The terms “stable”, and “stability” when referring to the disclosed composition, mean that an active component within, at a certain temperature and after certain time duration, remains at least 70% active, that is, capable of forming a fibrin clot. In some embodiments of any aspect of the composition disclosed herein, the composition may comprise one or more additives as described below, e.g., calcium salt and/or one or more excipients, e.g., selected from, without being limited thereto, one or more amino acids, albumin, saccharides, and/or saccharide derivatives.

The term “additive” is meant to be understood as any substance that can be added or originates from a source material to a composition and may also include an active additive such as calcium salt as described below. The term “excipient” as used herein denotes a non-active or non-therapeutic agent added to a pharmaceutical composition e.g., to provide a desired consistency or stabilizing effect.

Calcium is an important element in the clotting cascade and may be needed for activation of factor XIII into factor XIIIa, which cross-links and stabilizes fibrin to generate an insoluble clot. Calcium is also important for the generation of multiple enzymes in the coagulation cascade including generation of Factors IXa, Xa, and IIa, thus calcium is a critical component in the intrinsic pathway of clotting.

Accordingly, in some embodiments of any aspect of the disclosed composition, the composition further comprises an additive such as, without limitation, calcium. Calcium used with the invention may be in the form of a salt, e.g., calcium chloride salt. Alternatively, additional salts may be used, such as calcium acetate and/or calcium citrate.

### The Method of Treatment

In another aspect of the present invention, there is provided a method of treating a subject having a diseased or an injured tissue that can benefit from rapidly activating the intrinsic pathway of blood coagulation, the method comprising administrating to the subject a therapeutically effective amount of ellagic acid (EA) complexed with metal ions, the complex being incorporated within a buffer or a matrix comprising e.g., collagen or a derivative thereof.

Without being bound by any particular mechanism, to activate e.g., Factor XII, ellagic acid needs to be complexed with a metal ion to form an aggregate. This provides the surface for protein binding and activation.

By “treating a disease or an injured tissue” it is meant to encompass reducing blood loss at a bleeding site of a tissue (in vivo), e.g., in a patient undergoing surgery. The term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

Accordingly, in some embodiments, the method is for reducing blood loss at a bleeding site of a tissue and/or making a sealant layer in/on such a tissue, e.g., in a patient undergoing surgery, the method comprising contacting the disclosed composition in an embodiment thereof with the bleeding site and/or its proximity. Optionally, the method comprises first applying on such a tissue an aqueous solution, such as saline, and thereafter applying on the aqueous solution the disclosed composition, so as to promote a fast clotting-time. Accordingly, in some such embodiments, the composition, in any embodiment thereof, is for use as a hemostat in a bleeding site of a tissue.

As used herein, the term “subject” shall mean any animal including, without limitation, a human, a mouse, a rat, a rabbit, a non-human primate, or any other mammal. In some embodiments, the subject is human, e.g., a human patient. The subject may be male or female. In one embodiment, the subject in need is a patient.

The term “administrating” means providing a certain strain according to the present invention or a certain composition comprising the stain as an active ingredient to a subject by any suitable method. In some embodiments the complex is administered to the subject via a local administration route selected from topical, subcutaneous or intradermal administration.

In some embodiments, the administration is to the injured or bleeding site of an organ. The term “administrating to the site” (of the subject) means administrating to: (a) the place on the body surface, which is in correspondence with, or, close to the “site”; and/or (b) the place on the body surface that provides an accessible route to the “site”.

Toxicity and therapeutic efficacy of the active ingredients described herein may be determined by standard pharmaceutical procedures in vitro, in cell cultures or experimental animals. The data obtained from these in vitro and cell culture assays and animal studies may be used in formulating a range of dosage for use in human. The dosage may vary depending upon the dosage form employed and the route of administration utilized. The exact formulation, route of administration, and dosage can be chosen by the individual physician in view of the patient's condition (see, e.g., Fingl E. et al. (1975), “The Pharmacological Basis of Therapeutics”, Ch. 1, p. 1.).

Dosage amount and administration intervals may be adjusted individually to provide sufficient plasma levels of the active ingredient to induce or suppress the biological effect (i.e. coagulation activity). The coagulation activity will vary for each preparation, but can be estimated from in vitro data. Dosages necessary to achieve the coagulation activity will depend on individual characteristics and route of administration. Detection assays can be used to determine plasma concentrations.

Examples of dosage forms for topical administration include, but are not limited to, transdermal patch, cream, foam, gel, lotion, ointment, paste, powder, shake lotion, solid, sponge, tape, tincture, vapor, injection, drops, rinses, spray, and solution. A “unit dose” or “dosage unit” may be configured to provide a full unit dose or fraction thereof (e.g., or ¼ of a dose).

In some embodiments, the term “dose”, as used herein, means a drug or active component taken each time by an individual subject, in particular the total amount of a drug or active component taken each time by an individual subject, for one site.

In some embodiments, the term “dosage form”, as used herein, means a unit of administration of an active agent. Examples of dosage forms include tablets, capsules, injections, suspensions, liquids, emulsions, creams, ointments, suppositories, inhalable forms, and the like.

A predetermined quantity in each unit dose can depend on factors that include, but are not limited to, the unique characteristics of the active compound and the particular therapeutic effect to be achieved, and the limitations inherent in the art of creating and administering such unit doses. For instance, a unit dose may be, a transdermal patch, a spray, i.e., one spray in the spray application, a droplet of the dripping application, a certain length of the tape, rice-sized or bean-sized ointment, or a scoop or a spoon of ointment.

Unit dose measuring devices or kits, such as a cup, scoop, syringe, dropper, spoon, or colonic irrigation device, may hold the dosage form, for instance cream, foam, gel, lotion, ointment, paste, powder, shake lotion and solid, a measured quantity of composition equaling a full unit dose or fraction thereof (e.g., ½, ⅓ or ¼ of a dose). There may be a single unit dose, or multiple unit doses, in a single dose of administration. The kit may include instructions regarding the size of the unit dose, or fractions thereof.

In some embodiments, the EA complexed with metal ions is in a pharmaceutically acceptable form. In some embodiments, the EA complexed with metal ions is in dosage form.

In some embodiments, the terms “unit dose” or “dosage unit” refer to a dosage form that is configured to deliver a specified quantity or dosage of composition or component thereof.

Depending on the severity and responsiveness of the condition to be treated, dosing can be of a single or a plurality of administrations (e.g., weekly or bi-weekly administrations), with course of treatment lasting from several minutes to several hours, or until curing (i.e. clotting) is affected or diminution of the disease state is achieved.

The term “pharmaceutically acceptable” is defined herein to refer to those compounds, materials, compositions and/or dosage forms, which are, within the scope of sound medical judgment, suitable for contact with the tissues a patient without excessive toxicity, irritation allergic response and other problem complications commensurate with a reasonable benefit/risk ratio.

As used herein, the term “injured tissue”, (or “damaged tissue”) refers to disruption of the normal continuity of structures caused by a physical (e.g., mechanical) force such as in incisions caused by surgery, a biological (e.g., thermic or actinic force), or a chemical means. The term “injured tissue” also encompasses contused tissues, as well as incised, stabbed, or lacerated tissue, as well as open, puncture, and injuries caused e.g., by ripping, scratching, pressure, and biting. The term “injured tissue” also encompasses wounds and bleeding sites as described hereinthroughout.

The term “tissue” may refer to a tissue selected from a soft tissue and a bone tissue. The term “soft tissue” as used herein relates to a body tissue that is not hardened or calcified. This term especially relates to soft tissues that are vascularized and therefore may be a source of bleeding. Examples for such tissues include, but are not limited to, connective tissue (such as tendons, ligaments, fascia, skin, fibrous tissues, fat, and synovial membranes), muscles, and internal organs. In general, soft tissues are meant to exclude bone tissue.

Thus, in some embodiments, the disclosed composition in any embodiment thereof may be used or applied in difficult-to-reach bleeding sites, for example during minimally invasive surgeries (MIS) such as, e.g., endoscopy. One of the common forms of endoscopy is laparoscopy which is minimally invasive inspection and surgery inside the abdominal cavities.

The term “surgery” also encompasses the time during or after surgical or diagnostic procedures. Further non-limiting examples of procedures include neurosurgery, abdominal surgery, cardiovascular surgery, thoracic surgery, head and neck surgery, pelvic surgery and skin and subcutaneous tissue procedures. For at least one of these situations, the composition of the invention may serve as a suitable sealant, which may permit adhesion to the bleeding wound, and thus addresses hemostasis without being dependent on the condition of the wounded tissue, e.g., severity of bleeding. The formation of the sealant occurs in a relatively short period of clotting time subsequent applying the disclosed composition, in any aspect and/or embodiment thereof, on an injured tissue.

As shown in the Examples section below, in some embodiments, a blood clotting is affected within less than about 400 sec, upon the administrating or contacting the injured tissue with said matrix. In some embodiments, a blood clotting is affected within less than about 300 sec, upon the administrating or contacting the injured tissue with said matrix. In some embodiments, a blood clotting is affected within less than about 250 sec, upon the administrating or contacting the injured tissue with said matrix. Typically, after using 1 to 3 min tamponade, a partial clot may form which enables to stop bleeding, but more time may be needed for clotting reacting up to the paste surface thus forming a complete clot.

In another aspect of the present invention, there is provided a method of treating a wound comprising the step of applying (e.g., contacting) the disclosed composition in any aspect and/or embodiment thereof onto and/or into the wound of a subject in a need thereof. In some embodiments, the treatment is effected in vivo or locally, i.e. at a bleeding site. By “applied to a bleeding site of need” it is meant to refer, e.g., to a topical application of the composition at, or on/near a bleeding surgical site of a tissue.

The composition and the method of treatment disclosed herein may be used for any therapeutic purpose. The term “any therapeutic purpose” refers to any curative or preventive treatment in a subject. Exemplary therapeutic purposes include, but are not limited to, sealing a bore hole formed in a tissue or organ e.g., a bone; anastomosis at blood vessels; joining tissue parts e.g., soft tissue parts; treating or preventing dura defects e.g., tears and leaks following injections, fissures or cracks; treating or preventing bleeding; treating or preventing air leaks such as following pulmonary lung resection; treating or preventing defects following intestinal perforation; treating or preventing defects following anastomosis procedure carried out in any tissue e.g., uterine, esophagus, stomach, pancreas, pancreatic duct, gall bladder, bile duct, intestinal (including the small intestine and the large intestine), and rectum; treating or preventing post-operation leaks in any tissue e.g., uterine, esophagus, stomach, pancreas, pancreatic duct, gall bladder, bile duct, intestinal (including the small intestine and the large intestine), and rectum; preventing or diminishing the occurrence of post-operative leaks at the staple or suture line e.g., by applying the composition according to the invention, either alone or combined with an additional matrix e.g., a patch, or pad onto at least a part of a defect such as a staple/suture line; for strongly affixing prosthesis e.g., during a hernia operation; for staple/suture line reinforcement; to prevent or diminish air leak, e.g., alveolar air leakage; treating or preventing renal defects; treating or preventing fistulas; treating or preventing heart defects e.g., penetrating heart wounds; reinforcing of a vascular graft prosthesis; and treating or preventing cerebrospinal fluid leakage.

In some embodiments, the composition, complex or a matrix according to any of the embodiments disclosed herein is for use in providing hemostasis, sealing leaks and/or joining structures. In some embodiments, there is provided a method of providing hemostasis, scaling leaks and/or joining structures in a subject in need thereof, the method comprising use of the composition, the complex, or the matrix according to any of the embodiments disclosed herein.

The term “hemostatic” refers to an ability to prevent, reduce, or stop blood loss e.g., from wounds, such as surgical or traumatic wounds, or to assist in hemostasis e.g., by promoting blood clot formation. “Hemostasis” (or “haemostasis”) refers to the first stage of wound healing. It is a process which causes bleeding to stop. By “assist in hemostasis” it is meant to help reduce or stop bleeding. By “applied to a bleeding tissue” it is meant to refer to e.g., a topical application of the composition at the bleeding site, e.g., at a surgical site to control bleeding.

Control of bleeding is needed in various situations including treatment of wounds. As used herein, the terms “controlling”, “preventing”, or “reducing”, which may be used herein interchangeably in the context of the bleeding, including any grammatical inflection thereof, indicate that the rate of the blood extravagated is essentially nullified or is reduced e.g., by at least 10%, at least 20%, at least 30%, at least 40%, at least 50%, at least 60%, at least 70%, at least 80%, at least 90%, or even by 100%, of the initial rate of bleeding, compared to situation lacking the contact of the disclosed composition in/on the bleeding site. Methods for determining a level of appearance of bleeding are known in the art.

Further, in some embodiments, the terms “controlling”, “preventing”, or “reducing”, in the context of the bleeding are also meant to encompass at least partially sealing blood vessels.

As used herein, the term “bleeding” refers to extravasation of blood from any component of the circulatory system. “Bleeding” thus encompasses unwanted, uncontrolled and often excessive bleeding in connection with surgery, trauma, or other forms of tissue damage, as well as unwanted bleedings in a patient having bleeding disorders, and severe bleeding after birth, including hemorrhage associated with caesarian section. The term “blood”, or any grammatical inflection thereof, also includes blood fractions, such as plasma or red blood cells.

“Wound” as used herein refers to any damage to any tissue of a patient which results in the loss of blood from the circulatory system and/or any other fluid from the patient's body. The damage may have been caused by any agent or source, including traumatic injury, infection or surgical intervention. A wound may be in a soft tissue, such as an organ, or in hard tissue, such as bone. The tissue may be an internal tissue, such as an organ or blood vessel, or an external tissue, such as the skin. The loss of blood may be internal, such as from a ruptured organ, or external, such as from a laceration.

In some embodiments, the matrix comprises a combination of gelatin in the paste form and in the powder form.

Further embodiments described above under “The Composition”, e.g., of the polymeric matrix, tablet, powder, and gelatin are incorporated herein.

### Method of Making a Hemostatic Composition

According to another aspect of the present invention, there is provided a method of making a hemostatic composition comprising ellagic acid (EA) complexed with metal ions to an absorbable polymeric matrix, comprising the step of: adding to the matrix the complex.

The term “adding” or the like means contacting one reactant, reagent, solvent, or buffer in which the reagent or reactant (e.g., EA) are present. Reactants, reagents, solvents, catalysts, or the like can be added individually, simultaneously, or separately and can be added in any order.

In exemplary embodiments, the EA-metal ion (e.g., Ni) complex is incorporated first with first with gelatin powder followed by combining with gelatin paste.

As described in Example 2, combining EA-Ni complex with SURGIFLO® paste alone (34 μg EA-Ni/ml paste) result in four time less clotting potency (about 400 sec in a 4 ml whole blood gelation test) as opposed to combining it first with SURGIFOAM™ at the same EA-Ni complex concentrations (about 100 sec clotting time).

In some embodiments, the matrix may be added to the complex at any ratio described hereinabove under “The Composition”, e.g., in the range from 56,000:1 to 35:1, w/w, complex of polymeric matrix to EA-metal ion (e.g., nickel) complex, by weight, respectively, or, e.g., in the range of matrix to EA-metal ion (e.g., nickel) 5600:1 to 350:1, by weight, respectively.

Further embodiments described above under “The Composition”, e.g., of the polymeric matrix, tablet, powder, and gelatin are incorporated herein.

In some embodiments, the EA-metal ion (e.g., nickel) complex is suspended with the matrix (e.g., gelatin) powder before combining with a paste matrix of e.g., gelatin (see Example 2 below).

In some embodiments, there is provided hemostatic composition obtainable by any embodiment of the herein disclosed method of making a hemostatic composition. The term “obtainable” as used herein also encompasses the term “obtained”. In one embodiment, the term “obtainable” means obtained.

### The Kits and Additional Aspects and Embodiments

In some aspects, the disclosed composition, in any embodiment thereof, is provided in a kit. According to an aspect of the present invention, there is provided a kit comprising: a container containing ellagic acid (EA) complexed with metal (e.g., nickel) ions; a polymeric matrix; an applicator for applying the composition to a tissue. According to another aspect of the present invention, there is provided a kit comprising: a container containing EA; a container containing metal (e.g., nickel) ions; a polymeric matrix; an applicator for applying the composition to a tissue.

In some embodiments of this aspect, the EA complexed with metal ions is provided within a buffer.

In some embodiments of this aspect, the buffer comprises a solution having tetramethylammonium hydroxide (TMAH).

Compositions described herein, as well as the contents of the kits, may, if desired, be presented in a pack or dispenser device, which may contain one or more unit dosage forms containing the composition or ingredients and/or reagents (e.g., a buffer or an aqueous solution) for preparing the composition. The pack may, for example, comprise metal or plastic foil, such as a blister pack. The pack or dispenser device may be accompanied by instructions for administration. The pack or dispenser device may also be accompanied by a notice in a form prescribed by a governmental agency regulating the manufacture, use, or sale of pharmaceuticals, which notice is reflective of approval by the agency of the form of the compositions for human or veterinary administration. Such notice, for example, may include labeling approved by the U.S. Food and Drug Administration (FDA) for prescription drugs or of an approved product insert. Compositions comprising a preparation of the invention formulated in a pharmaceutically acceptable carrier may also be prepared, placed in an appropriate container, and labeled for use for an indicated application and/or for treatment of an indicated condition, as further detailed herein. The term “preparation” refers to physiologically suitable for therapeutic use.

In some aspects, there is provided a method of activating the intrinsic pathway of blood coagulation, the method comprising administrating to the subject a therapeutically effective amount of ellagic acid (EA) complexed with metal e.g., nickel, ions, optionally, the complex being incorporated within a matrix comprising collagen or a derivative thereof or within an appropriate buffer as described herein.

It will be appreciated that compositions of embodiments of the present disclosure may be attached to or included in medical devices, such as for promoting wound healing.

In another aspect of the present disclosure, the disclosed composition in any aspect or embodiment thereof is for use in a method for preparing a fibrin sealant in/on an injured tissue of a subject, e.g., by applying the disclosed composition in any aspect and/or embodiment thereof on or in a proximity to a surface of the tissue.

The term “fibrin” does not only refer to fully coagulated fibrinogen but further includes any mixture of fibrin and fibrinogen which may occur during formation of fibrin from fibrinogen using thrombin and, thus, includes any ratio of fibrinogen/fibrin and any grade of gelation and/or clotting conceivable as long as it has no negative impact on the final pasty texture of the composition. Moreover, the term “fibrin” further includes any partly or fully cross-linked, gelled or clotted form.

In the context of the present invention, the term “sealant”, also referred to as “fibrin sealant”, and “biological glue”, is to be understood as an adhesive, glue, or hemostat, e.g., such as one originates or being derived from the disclosed composition, having ingredients that upon contact with, or in proximity to, a tissue and/or blood, reacts to subsequently form a clot, and may further act as a tissue adhesive, and thereby prevents, reduces, or stops bleeding, joint structures and/or seals physiological leaks, e.g., of cerebrospinal fluids (CSF), lymph, bile, gastrointestinal (GI) content, air leak from lungs etc.

It is thus to be appreciated that fibrin per se exists as a polymeric, insoluble matrix comprising fibrils, each fibril comprising many fibrin molecules.

In another aspect of the present disclosure, there is provided a method for preparing a fibrin sealant in/on an injured tissue of a subject, e.g., by applying the disclosed composition in any aspect and/or embodiment thereof as a hemostat on a surface of the tissue.

According to another aspect of embodiments of the present invention, a kit is provided for generating a composition described herein. It is appreciated that the consistency of the composition is such that it can be applied, for example, by spreading or by sticking the composition directly onto a damaged tissue and/or on a bleeding site. Accordingly, the composition does not need to be further spread on or applied to a solid surface, object, or other solid medium such as a strip or a film in order to be in the appropriate form for applying onto a damaged tissue or bleeding site. Nevertheless, a suitable applicator, such as, for example, a syringe, may be used in order to apply, spread or stick the composition onto the bleeding site, for the purpose of easy access and handling.

In an additional aspect and/or embodiment, the present invention provides a kit comprising: a) a container containing a composition of the invention as described above, b) an applicator for applying the composition to a tissue, and c) optionally instructions for use. In another embodiment, the present invention further provides a hemostatic kit comprising a container containing the herein disclosed composition in an embodiment thereof.

Additionally, or alternatively, the hemostatic kit may comprise a syringe containing the blend, mixture or powder and another syringe containing the dispersant. For example, dual-syringe mixing devices may produce a substantially homogenous paste mixture by combining initially separate liquid such as dissolved EA and powders such as metal (e.g., nickel) salt and then passing the blended contents back and forth between two connected syringes via interconnected outlet(s). Therefore, a low expression force for dispensing the paste from a syringe may be preferred for ease of mixing and ultimately for deployment of the resulting paste. The desired expression force may be less than 1.51 lbf.

In some embodiments, at least one of the containers in the kit is a pre-filled syringe. In some embodiments, a syringe is provided in addition to the container(s) of the kit. In some embodiments, the container is in a specific type, such as a vial or an applicator such as syringe. In some embodiments, a syringe is provided in addition to the container(s) of the kit. The term “container” may refer to any generic structure such as a vessel or a vial.

The kit may be applied using an applicator device which may be used for administering several and sequential injections of the composition. In one embodiment, the applicator device enables multiple injections of a fixed-dose of the mixed components on a 2-D surface of a tissue while moving the device. In one embodiment, the applicator has a syringe with an injection needle, which is optionally automatically retracted from the patient's body organ after the injection is completed without the need for the administrator to lift the device upward from the injection surface. In one embodiment, the kit may be used for the administration of a sealant.

The hemostatic kit of the invention may be a kit for use in reducing, preventing or stopping blood flow, e.g., in open wounds, and it may be used for reducing, preventing or stopping blood flow during a procedure, such as during, before, or after a surgical procedure such as, for example, laparoscopic surgery, neurosurgery, abdominal surgery, cardiovascular surgery, thoracic surgery, head and neck surgery, pelvic surgery and skin and subcutaneous tissue procedures. The kit may be used for reducing or preventing blood flow from the skin, or in internal organs.

In some embodiments of any aspect of the kit and/or compositions, the composition is sterile. Especially when handling blood products, the sterility issue is crucial, and specifically the issue of viral inactivation. In general, viral inactivation may be carried out by any number of methods, including solvent detergent, heat inactivation, irradiation, and nanofiltration. Typically, the standard for viral inactivation requires using two different methods. Additionally, FDA standard for sterility requires filtration.

Any aspect and embodiment of the herein throughout disclosed composition may be incorporated to the aspect and embodiments of the methods, and the kit, mutatis mutandis.

### General

The terms “comprises”, “comprising”, “includes”, “including”, “having”, and their conjugates mean “including but not limited to”. The term “consisting of” means “including and limited to”. The term “consisting essentially of” means that the composition, method or structure may include additional ingredients, steps and/or parts, but only if the additional ingredients, steps and/or parts do not materially alter the basic and novel characteristics of the claimed composition, method or structure.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

The word “optionally” is used herein to mean “is provided in some embodiments and not provided in other embodiments”. Any particular embodiment of the invention may include a plurality of “optional” features unless such features conflict.

As used herein, the singular form “a”, “an” and “the” include plural references unless the context clearly dictates otherwise. For example, the term “a compound” or “at least one compound” may include a plurality of compounds, including mixtures thereof. In those instances where a convention analogous to “at least one of A, B, and C, etc.” is used, in general such a construction is intended in the sense one having skill in the art would understand the convention (e.g., “a composition having at least one of A, B, and C” would include but not be limited to compositions that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B”.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques, processes, and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition or substantially preventing the appearance of clinical or aesthetical symptoms of a condition.

It is appreciated that certain features of the invention, which are, for clarity, described in the context of separate embodiments, may also be provided in combination in a single embodiment. Conversely, various features of the invention, which are, for brevity, described in the context of a single or some embodiments, may also be provided separately or in any suitable subcombination or as suitable in any other described embodiment(s) of the invention. Certain features described in the context of various embodiments are not to be considered essential features of those embodiments, unless the embodiment is inoperative without those elements.

The terms “about” or “approximately” as used herein mean that values that are 10% above or below the indicated value are also intended to be included. Unless otherwise indicated, all numbers such as those expressing, for example, ratios, weight, mole/mole, amounts, viscosity, temperatures, etc., are to be understood as being modified in all instances by the term “about”. Accordingly, unless indicated to the contrary, the numerical parameters set forth in this description and attached claims are approximations that may vary by up to plus or minus 10% depending upon the desired properties sought to be obtained by the present invention.

Various embodiments and aspects of the present invention as delineated hereinabove and as claimed in the claims section below find experimental support in the following examples.

### EXAMPLES

Reference is now made to the following examples, which together with the above descriptions illustrate some embodiments of the invention in a non-limiting fashion.

### Example 1—Clotting Time Assays With Various Activators

Exemplary procedures were conducted to evaluate various hemostatic agents that act through the natural physiologic pathway of coagulation. The aim was to assess how quickly the activators clot fresh whole blood. A couple of test methods were developed to assess the activators (i) Actalyke Activated Clotting Time (ACT) analyzer (manufactured by Helena Laboratories) and (ii) Whole Blood (WB) gelation test.

These clotting time assays evaluate the ability of a material to active the intrinsic pathway to form a fibrin clot in whole blood after recalcification of citrated blood. Fresh citrated blood is used to preserve clotting factors. These tests are well suited for particulate activators, e.g., Celite, Kaolin, Bioglass.

ACT test: Once the analyzer was switched on and equilibrated to 37° C., biological control plasmas were evaluated with celite to confirm the operation of the analyzer. The general procedure was to combine the activator into an empty ACT tube (containing only a magnet) with citrated bovine blood (2 mL) followed by immediate mixing. After mixing blood and activator, 1.0 mL calcium saline solution (25 mM CaCl₂ in normal saline, 0.9% sodium chloride solution) was added to the tube and inserted into the analyzer. The clotting time was detected based on the displacement of a magnet. It is noteworthy that since the ACT is designed for small particulate activators, e.g., celite and kaolin, larger biomaterials (like gelatin particles) cannot mix adequately. This is the reason the WB gelation test was mostly used for testing the gelatin formulations.

WB Gelation Test: The WB gelation test uses a larger volume of blood compared to a small volume ACT tube to accommodate biomaterials, e.g., gelatins, collagen, thus allowing the WB gelation test to assess interactions between activators and biomaterials. The Actalyke ACT analyzer was designed for testing small particulate activators like Celite and Kaolin (used for clinical applications). However, when the amount of activator or biomaterial (e.g., gelatin) is too large, the analyzer produces errors. To address this limitation, the WB Gelation Test was developed with a heated enclosure and larger tubes to accommodate larger volumes of blood and greater amounts of activators. Setup includes a plexiglass enclosure with a heating unit to perform the testing at 37° C. A temperature sensor in the enclosure maintained the temperature at 37° C. ±1° C. The enclosure contains an orbital rotator to gently mix the blood to with the other materials being tested while keeping it warm. The calcium saline and tube rocker are also in the enclosure. The enclosure has a front hinge to allow access from the front of the unit.

The stability of the WB was assess using the ACT. Twelve mg of celite was added to an empty tube (containing only a magnet) and mixing immediately with citrated bovine blood (2 mL). After mixing blood and activator, 1.0 mL of the calcium saline solution was added to the tube and inserted into the analyzer. The rate of clot formation was determined based on displacement of the magnet. The goal of this testing was to show that the WB was stable over the course of a day, thus showing that the blood does not change, e.g., clotting factor degradation, and measures taken on a single day are comparable.

In exemplary procedures, citrated whole blood (4 ml) was added to a 15 ml conical tube with activator/biomaterial and mixed, then calcium saline solution (2 ml) was added to the tube and the timer is started. A heated enclosure was used to be comparable to in vivo or ex vivo studies. The tube had been placed on a rocker and observed until above 90% clot formation was observed. Multiple replicates were tested for each activator.

In Exemplary procedures, bioactive glass particles were tested with fresh whole blood. Celite was tested as a positive control (Celite was purchased from EP Minerals, Reno, USA, Product code EP-300, Diatomite filter media) and bioactive glass was from MO-SCI, Rolla, USA, 45S5 (Bioglass material, also called “Bioglass 45S5” or “calcium sodium phosphor silicate” is a bioactive glass specifically composed of 45 wt % SiO₂).

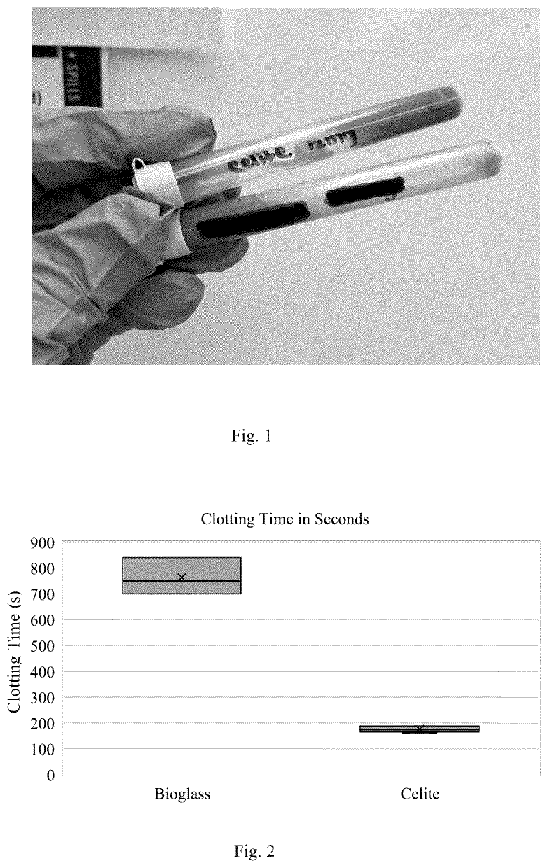

In the ACT test, 45 mg and 65 mg Bioglass in 2 mL of blood were tested. These samples did not clot at all or it took an extended time to clot (i.e. the blood freely flowed in the tube after more than 4 minutes). FIG. 1 presents a photographic image showing recalcified blood after several minutes with Celite particles, 12 mg, (clotted in tube; top). FIG. 2 shows comparative ACT data for Celite (12 mg) and bioactive glass (45 mg). Celite particles clotted the blood rapidly (under 200 seconds), while the clotting time was longer for bioactive glass particles which clotted the blood within a range of 700-840 seconds. Bioactive glass was tested over a range of concentrations (12-65 mg) with no improvement in clotting time, data not shown.

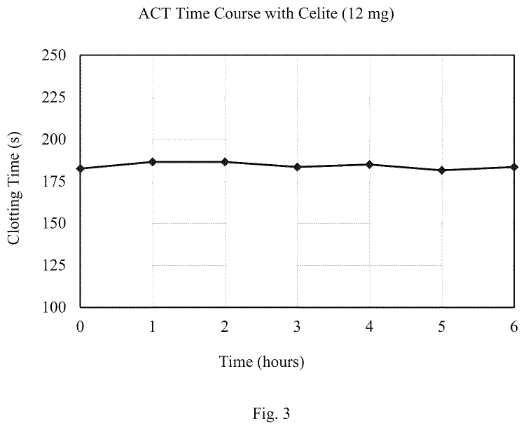

FIG. 3 presents ACT Time Course with celite, showing the time course over a day's time testing celite (12 mg) over the course of 6 h in the ACT analyzer. These data show that the WB was stable for a 6 h testing duration, the limit for whole blood usage.

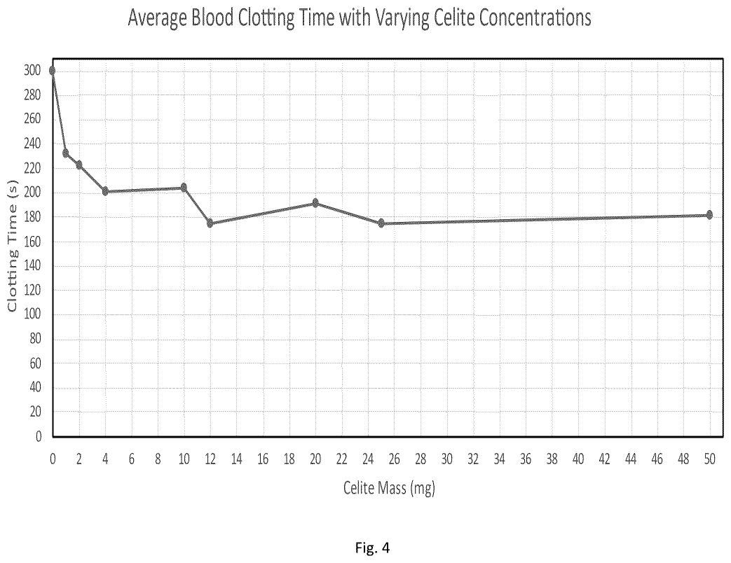

FIG. 4 presents ACT data with varying Celite concentrations. The celite dose response was evaluated by combining various amounts of celite (0-50 mg) and 2 mL of citrated bovine whole blood into an empty tube (containing only a magnet). After mixing blood and activator, 1.0 mL of calcium saline solution was added to the tube and inserted into the ACT analyzer. The rate of clot formation was determined. These data show that the WB clotting time decreased between 0-12 mg of celite and then leveled off such that no acceleration of clotting time was observed after 12 mg of the activator.

In additional exemplary procedures, medium and long chain polyphosphate were compared to celite for its ability to clot whole blood. Two amounts (12 mg and 20 mg) of medium and long chain polyphosphate (purchased from Kerafast, Boston, USA, P100 and P700 polyphosphate) were evaluated by combining with 2 mL of citrated bovine whole blood in an empty tube (containing only a magnet). After mixing blood and activator, 1.0 mL of the calcium saline solution was added to the tube and inserted into the ACT analyzer. The rate of clot formation was determined. Amounts of 12 mg of stock celite (from EP Minerals, Reno, USA, Product code EP-300, Diatomite filter media) and 14 mg of Helena celite (from Helena Laboratories) were tested in the same study. The medium and long chain polyphosphate groups had mean clotting times ranging from 288 seconds to 600 seconds, while the mean clotting times for the celite group were 197 seconds and 218 seconds for the stock celite and Helena celite, respectively.

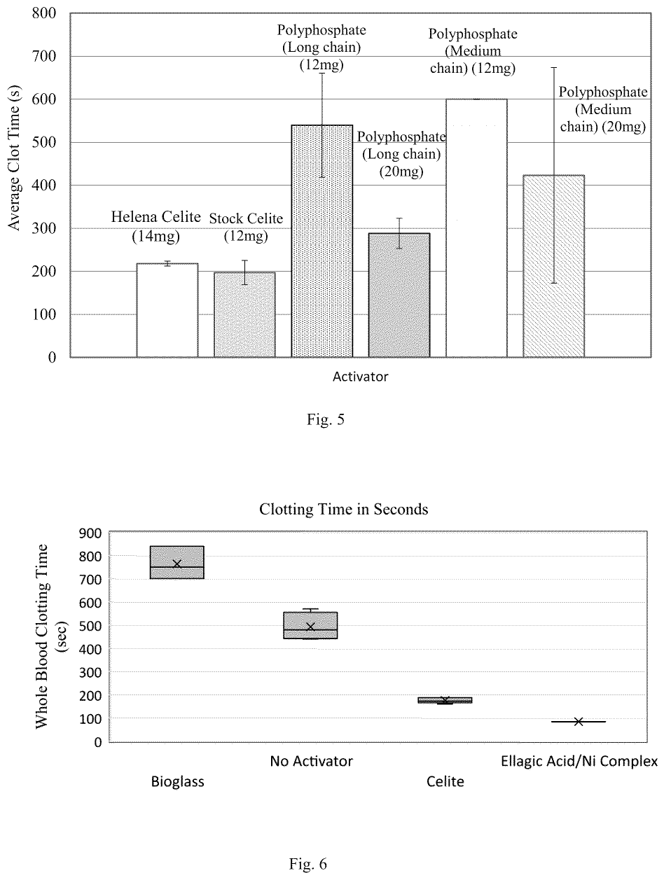

FIG. 5 presents ACT Results with various types of polyphosphate and Celite. These data show that the WB clotting time of the celite activators were significantly faster than either medium and long chain polyphosphate (both at 12 mg and 20 mg) demonstrating that celite is a superior activator in this WB clotting assay.

### Ellagic Acid

EA-Ni complex preparation: 1.8 mL 10% (w/v) tetramethylammonium hydroxide (TMAH; purchased from Sigma Aldrich) was dissolved in 250 ml of water. An amount of 0.034 g ellagic acid (ellagic acid hydrate; purchased from Sigma-Aldrich; Product number 372749; EA molecular weight is 302.2 g/mole, which gives 0.0001125 moles EA) was then dissolved in this TMAH solution, followed by 1.5 g phenol dissolved in the TMAH-ellagic acid solution, whereafter 1.0 ml 0.1 M nickel chloride (purchased from Sigma Aldrich; i.e. 0.0001 moles Nickel) was added, and the resulting solution mixed for 10 minutes with a Teflon coated stirring bar on a stir-plate. An amount of 1.2 g TRIS-HCl was then dissolved into the solution. EA-Ni complex was tested in the whole blood gelation test.

Testing the EA-Ni complex in the WB Gelation Test at a concentration of 0.085 mg/mL yielded impressive results. Whole blood clotting times were significantly faster (mean 84 sec) compared with that of 24 mg of celite (mean 173 s). Ellagic acid without Nickel (or any other metal) was tested but not clotting was observed over 1200 minutes. Bioactive glass (as described above) produced a clotting time of 763 sec. Comparative results are shown in box plots, FIG. 6. It is noteworthy that EA-Copper complex was also tested, but the clotting times were significantly 4×'s longer (data not shown), though still faster than the bioactive glass.

### Ellagic Acid Dose Response

In exemplary procedures, a dose response study was performed with EA-Ni complex, as detailed in FIG. 7A showing mean values of triplicate results at each concentration tested. Various amounts of the EA-Ni complex were tested in the WB Gelation test. The results showed that there was a broad range of EA-Ni complex concentrations that could rapidly clot the blood. EA-Ni complex was therefore proved as a very efficient activator with as little as about 2 μg/mL EA-Ni complex in blood enabling rapid activation (approximately 100 sec). Even at levels as low as 2 μg/mL EA-Ni complex in blood, it was clotting the whole blood at a rate similar to 24 mg celite. Without activator, blood did not clot in 900 sec. Ellagic acid without Nickel (or any other metal) was tested but no clotting was observed over 1200 minutes. There is no statistically significant trend (p=0.944) in mean clotting times for EA-Ni complex concentrations in blood between 0.002125 to 0.034 mg/mL (2 to 34 μg/mL which can serve as a preferred range of EA-Ni) demonstrating the broad range of EA-Ni complex concentrations capable of rapidly activating clotting, see FIG. 7B showing that there is no significant trend in clotting times for EA-Ni complex concentrations in blood between 0.002125 to 0.034 mg/mL.

### Ellagic Acid Nickel Complex With Supplemental Phospholipid

Another exemplary set of experiments was carried out to determine whether the clotting rate can be improved with added phospholipid, that is, platelet/phospholipid dependency. The goal was to determine the criticality of platelets and whether the clotting rate can be further improved with added phospholipid. The EA-Ni was tested with whole blood and with cephalin added.

The EA-Ni complex was tested with whole blood and blood supplemented with cephalin using the WB gelation test procedure described previously. Cephalin is a rabbit brain-derived coagulation reagent containing a mix of phosphatidylserine (PS), phosphatidylethanolamine (PE) and phosphatidylcholine (PC), suitable for coagulation assays (the cephalin is made by BioData corporation, and it is a reagent to used perform a non-activated aPTT. The cephalin was used in its undiluted form in this test system, 1×0 and 5× of the standard amount were used).

The results are presented in FIG. 8. When a small volume of cephalin (0.1 mL) was added to 4 mL of blood, the mean clotting time with EA-Ni complex was 89 s, compared with a mean clotting time of 87 s without any added cephalin. With a larger volume of cephalin (0.5 mL) added to the 4 mL of blood, the mean clotting time remain unchanged at 87 s, that is, no improvement in clotting time was observed with added phospholipid to fresh whole blood.

### Ellagic Acid Nickel Complex Inhibited With Corn Trypsin Inhibitor (CTI)

The goal of this testing is to confirm that the mechanism of action of the EA-Ni complex is via FXII activation by using corn trypsin inhibitor (CTI), an inhibitor of FXIIa. The CTI was purchased from Prolytix (formerly Haematologic Technologies).

Using the WB gelation test procedure described previously, 0.1 mL of 2.5 mg/mL CTI was added to 4 mL of blood, the mean clotting time with EA-Ni complex was 141 s, compared with a mean clotting time of 87 sec without any added CTI. To further confirm the effect of the inhibitor, a larger amount of CTI (0.5 mL of 2.5 mg/mL) was added to the blood and the resulting clotting time was substantially longer (mean of 407 s).

The results are presented in FIG. 9 showing that at low concentrations of CTI, there was a slight prolongation in clotting time, and at high concentrations, the impact on clotting time was dramatic. This confirms that Ellagic acid-Nickel complex is acting through the intrinsic pathway of coagulation.

### Example 2—Testing of Ellagic Acid Nickel Complex With Flowable Geatin

### In Vitro Model

SURGIFLO®/SURGIFOAM™ EA-Ni flowable paste preparation: 15 ml of the above-mentioned Ellagic Acid Ni Complex solution was added to a 1 g SURGIFOAM™ and reconstituted per the SURGIFOAM™ IFU. A syringe containing 8 mL of the SURGIFOAM™/Ellagic Acid Ni paste was mixed with a prepared syringe of 8 mL SURGIFLO® gelatin suspended in saline. The 8 mL SURGIFOAM™/Ellagic Acid Ni paste and 8 mL of SURGIFLO® gelatin were well mixed by syringe exchange (e.g., 6-to-mix process, see SURGIFLO® Hemostatic Matrix Kit simple preparation). This process involves rapidly passing the paste formulations between syringes to create a homogenous paste.

It was found that a SURGIFLO®/SURGIFOAM™ mixture with Ellagic Acid-Nickel complex yields rapid in-vitro clotting times, as well as fast/effective hemostasis in an in-vivo porcine spleen biopsy bleeding model. It was found that suspending the EA-Ni complex with SURGIFOAM™ powder before combining with SURGIFLO® is important for maintaining good potency of the EA-Ni complex. More specifically, combining EA-Ni complex with SURGIFLO® paste alone (34 μg EA-Ni/ml paste) result in four time less clotting potency (about 400 sec in a 4 ml whole blood gelation test) as opposed to combining it first with SURGIFOAM™ at the same EA-Ni complex concentrations (about 100 sec clotting time). It was also found that when prepared as described herein, 1 ml of the SURGIFLO®/SURGIFOAM™/EA-Ni complex (85 μg EA-Ni/ml paste) formulation was also capable of clotting 4 mL of whole blood gelation test in about 100 sec. For reference, the 1 mL of the EA-Ni complex solution yields whole blood gelation test times of approximately 75-90 s in the absence of any gelatin paste. 1 ml of SURGIFLO® or 1 ml of reconstituted SURGIFOAM™ (with Saline) were used as a negative control in the whole blood gelation test that resulted in no blood clotting after 1200 sec. It is not known why SURGIFLO® appears to deactivate the EA-Ni complex in in vitro model, while preparing the complex with SURGIFOAM™ does not.

In view of the above it can be concluded that EA-Ni complex can be used as a potent activator of the intrinsic coagulation pathway even at low concentrations. EA-Ni can be combined with SURGIFOAM™ powder and then with SURGIFLO® to create a gelatin paste with excellent in vitro and in vivo clotting properties. The flowability and consistency properties of the EA-Ni gelatin paste were similar to conventional gelatin paste, e.g., SURGIFLO®.

The presence of nickel might be a concern, however, the amount would be below the guideline for elemental impurities (ICH Q3D) limit of 20 μg/day. It can also be noted that TMAH (tetramethylammonium hydroxide) is a solvent used during manufacturing which can be purchased in a diluted version at safety.

### In Vivo Model

Preliminary studies showed that a combination SURGIFOAM™ powder and SURGIFLO® (gelatin formulations) can be combined with EA-Ni complex to create a formulation that can rapidly achieve hemostasis in the animal model. To prepare the EA-Ni complex paste used for in vivo testing, 4× Ellagic Acid Ni Complex solution was prepared as per Example 2 of European Patent 0525035 B1, except that 250 mL deionized water was used instead of 1 L to prepare a 4× solution. First, SURGIFOAM™/Ellagic Acid Ni complex was prepared by combining 15 ml of 4× Ellagic Acid Ni Complex solution with 1 g of SURGIFOAM™ powder and reconstituted per IFU by shaking vigorously. Next, 8 mL of the SURGIFOAM™/Ellagic Acid paste was transferred to a 20 mL syringe and in another syringe 8 mL of SURGIFLO® paste prepared with saline according to the IFU. The contents of the two syringes were mixed by syringe exchange until visibly homogenous. Taken together, the total gelatin weight in the total volume (16 mL) of SURGIFOAM™/SURGIFLO® paste is 1.533 g. The total weight of EA-Ni is 1.088 mg. Thus, the weight ratio of gelatin to EA-Ni is 1409:1, w/w. It is noteworthy that EA-Ni can be diluted 1:40 and still accelerate clotting (i.e. less than 200 s) based on the dose response studies. Calculating the preferred range of the weight ratio of gelatin to EA-Ni would therefore be 56,000:1 to 35:1. Also, EA-Ni can be diluted 1:4 to rapidly accelerate clotting (i.e. approx. 100 sec) based on the dose response studies. Calculating another preferred weight range of the ratio of gelatin to EA-Ni would be 5600:1 to 350:1.

A biopsy punch model was used to create bleeding injury (6 mm diameter×3 mm deep) in the spleen. The flowable gelatin with EA-Ni complex performed very well with hemostasis times comparable to SURGIFLO® with thrombin as described below. The mean hemostasis times were not statistically different at 173.0 s and 173.4 s for SURGIFLO®+SURGIFOAM™+EA-Ni and SURGIFLO®+Thrombin, respectively.

In exemplary procedures, after the target bleeding site was dried as much as possible, enough product (approximately 2 mL) was then applied to the site to cover the entire bleeding area. A wet gauze/pad may be applied immediately to hold the product in contact with the bleeding area. Initial tamponade pressure was then held for 2 minutes.

The gauze/pad was then gently lifted to inspect the area. A hemostasis confirmation evaluation was performed for up to an additional one minute. If no free flow bleeding was observed during this one-minute observation period, the time to hemostasis was noted as the time when tamponade was discontinued.

However, if free flow bleeding was observed following tamponade, additional test article may be applied at the discretion of the surgeon, and tamponade pressure was immediately reapplied for an additional 30-second period. Observation and treatment periods continue in this manner until either hemostasis was achieved or until the testing period reached five minutes. At five minutes, if bleeding was still persisting, the trial was aborted as a failure and recorded as “>5:00” (greater than five minutes).

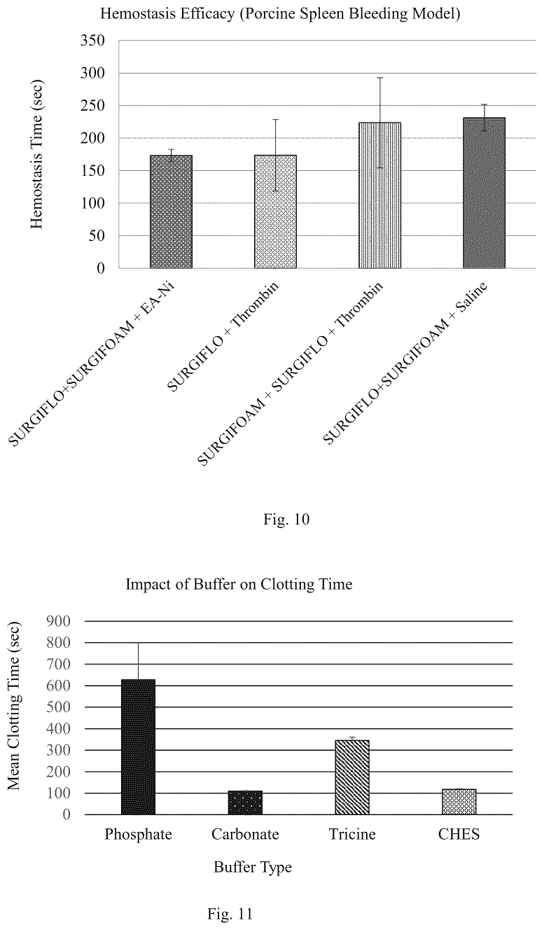

The results are presented in FIG. 10. It is shown that in the porcine spleen biopsy punch model, the above formulation (SURGIFLO®/SURGIFOAM™ 85 μg EA-Ni/ml paste as described for the in vitro tests) resulted in comparable Time to Hemostasis (TTH) as a standard SURGIFLO® with Thrombin (170-173 sec). The negative control, SURGIFLO®/SURGIFOAM™ (“SF/SFm”) paste, resulted in TTH of about 231 sec. There was a statistically significant difference in the mean hemostasis time for the SF/SFm+EA-Ni versus SF/SFm (t-test, p value 0.001). Another reason for preferring using SURGIFLO®/SURGIFOAM™ formulation for the in-vivo study was to provide an optimal paste consistency when testing in the animal model.

Flowable Paste with Buffering Agents: flowable gelatin paste formulations (SURGIFLO®) were combined with EA-Ni complex in the absence of gelatin powder. The EA-Ni complex formulation was the same as that described for the previous in vitro tests. To evaluate the role of various buffering agents, a 1 mL solution containing either phosphate, bicarbonate, tricine or CHES buffer at a concentration of 1 M was combined and mixed with 8 mL of gelatin paste containing 1.5 mL of EA-Ni complex by exchanging the contents rapidly between two syringes. All buffers were reagent grade and purchased from Sigma Aldrich. The gelatin paste with each buffer was tested in triplicate using the WB gelation assay as described previously.

There were significant differences in clotting times among the buffers. The mean clotting times of the phosphate, bicarbonate, tricine or CHES buffer were 627.3 sec, 109.3 sec, 345.3 sec and 117.7 sec, respectively, see FIG. 11. As a reference, the mean clotting time achieved with the SURGIFLO®/SURGIFOAM™/EA-Ni complex formulation described previously was approximately 100 sec in the WB gelation test.

### Example 3: Ellagic Acid Nickel Complex Lyophilized Tablets

In additional to the ellagic acid nickel complex being incorporated into a paste, the EA-Ni complex can also be imbibed or coated onto a sponge-like tablet matrix as a carrier. Dengen Dental Dengofoam gelatin sponges (1 cm×1 cm×1cm) were used as a carrier.

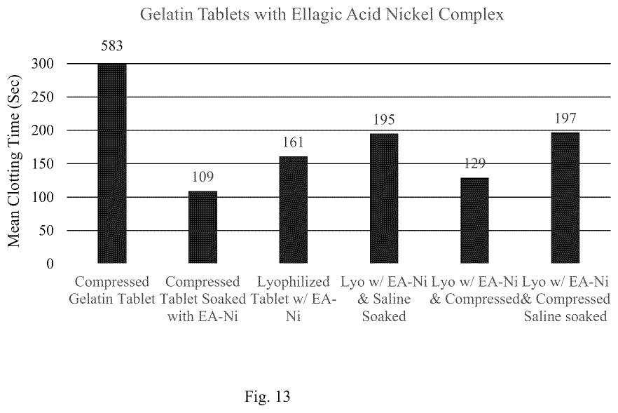

Exemplary procedures were performed as follows: for some samples, the sponge tablets were compressed in 1 dimension to a size of 0.2 cm×1 cm×1cm using a Dulytek DW8000 press. Uncompressed and compressed gelatin sponge tablets were soaked in 1 mL of 0.136 mg/ml EA-Ni complex (representing 4-fold (4×) higher concentration than described per Example 2 (Page 11) of EP Patent 525035 B1). Some of the soaked sponge tablets were lyophilized using a 4-day cycle to remove water from the EA-Ni sponge. Photos were captured for uncompressed tablets before and after soaking in EA-Ni, see FIG. 12A, and compressed tablets before and after soaking in EA-Ni, sec FIG. 12B.

Whole Blood gelation testing of Sponge Tables: using the WB gelation test procedure described previously, tablets with and without EA-Ni complex were tested by adding them to a tube of 4 mL of blood that was recalcified. The clotting time of a compressed gelatin tablet without EA-Ni was 583 sec. The mean clotting time was markedly shorted to 109 sec when the compressed tablet was soaked in EA-Ni complex. The tablets could also be lyophilized for preservation and long-term storage. Compressed and non-compressed lyophilized gelatin tablets had mean clotting times of 129 sec and 161 sec, respectively. The results are presented in FIG. 13. In can be concluded that soaking gelatin tablets with EA-Ni resulted in dramatic shortening of the whole blood clotting time. Tablets soaked in EA-Ni followed by lyophilization also substantially reduced the clotting time. Thus, the EA-Ni remained active after lyophilization. Interestingly, tablets soaked in EA-Ni followed by lyophilization and compression had shorter clotting times than similar uncompressed tablets. On the other hand, if an EA-Ni lyophilized tablet is first soaked in saline for 30 sec and then added to the whole blood, the clotting time is longer.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

1. A hemostatic composition comprising ellagic acid (EA) complexed with metal ions, said complex being dispersed, or embedded within an absorbable polymeric matrix.

2. The composition of claim 1, wherein the polymeric matrix comprises collagen or a derivative thereof.

3. The composition of claim 2, wherein the derivative of the collagen comprises gelatin.

4. The composition of claim 1, wherein the metal is selected from the group consisting of nickel, cobalt and copper.

5. The composition of claim 1, wherein the metal is nickel.

6. The composition of claim 1, wherein the metal ions and the EA are present at a molar ratio ranging from 100:1 to 1:100 metal to EA, optionally 10:1 to 1:10 molar ratio of metal ions to EA.

7. The composition of claim 1, wherein the absorbable polymeric matrix e.g., gelatin is in the form selected from a flowable paste at room temperature, a sponge-like matrix, a powder, and a combination thereof.

8. The composition of claim 1, being in the form of a paste.

9. The composition of claim 7, wherein said paste is characterized by a viscosity ranging from 1000-5000 Pa.s.

10. (canceled)

11. The composition of claim 23, wherein the matrix comprises a combination of gelatin in the paste form and in the powder form.

12. The composition of claim 1, being substantially devoid of thrombin.

13. The composition of claim 1, being substantially devoid of one or more coagulation factors selected from the inactive or active forms of Factors II, V, VII, VIII, IX, X, XI, XII and fibrinogen.

14. The composition of claim 1, wherein the metal ions are present at a preferred concentration ranging from 11,200:1 to 7:1, w/w, optionally, 1120:1 to 70:1 matrix, e.g., gelatin, to metal ion.

15. The composition of claim 1, wherein the EA complex is present at a concentration ranging from 56,000:1 to 35:1, w/w or 5600:1 to 350:1, w/w, optionally, by from 5600:1 to 350:1, w/w of the matrix, e.g., gelatin, to EA-Ni.

16. A method of treating a subject having a disease or an injured tissue that can benefit from rapidly activating the intrinsic pathway of blood coagulation, the method comprising administrating to the subject a therapeutically effective amount of ellagic acid (EA) complexed with metal ions, the complex being incorporated within a matrix comprising collagen or a derivative thereof.

17. The method of claim 16, wherein the administrating comprises contacting the injured tissue with said matrix.

18. The method of claim 16, wherein the metal is nickel.

19. (canceled)

20. (canceled)

21. The method of claim 16, wherein the derivative of the collagen comprises a combination of gelatin in the paste form and in the powder form.

22. (canceled)

23. (canceled)

24. (canceled)

25. (canceled)

26. (canceled)

27. (canceled)

28. A kit comprising:
(a) a container containing ellagic acid (EA) complexed with metal ions;
(b) a polymeric matrix;
(c) an applicator for applying the composition to a tissue; and optionally
(d) instructions for use.

29. The kit of claim 28, wherein the polymeric matrix comprises collagen or a derivative thereof.

30. The kit of claim 28, wherein the derivative of the collagen comprises gelatin.

31. The kit of claim 28, wherein the metal is nickel.

32. (canceled)

33. The kit of claim 28, wherein the EA complexed with metal ions is provided within a buffer.

34. The kit of claim 33, wherein the buffer comprises a solution having tetramethylammonium hydroxide (TMAH).
